(19) United States
(12) Patent Application Publication
(10) Pub. No.: US 2021/0378867 A1
(43) Pub. Date: Dec. 9, 2021

(54) APPAREL SYSTEMS AND METHODS FOR APPLYING TOPICAL TREATMENTS

(71) Applicant: Thomas Jefferson University, Philadelphia, PA (US)

(72) Inventors: Elizabeth Rose Schoenberg, Bala Cynwyd, PA (US); Stefani Lynne Samuels, Ambler, PA (US); Vivek Koratkar Bilolikar, Maple Grove, MN (US); Jennifer Lauren Doto, Philadelphia, PA (US); Matthew James Sharayko, Harleysville, PA (US); Matthew Scott Keller, Chadds Ford, PA (US); Mark Andrew Sunderland, Philadelphia, PA (US)

(21) Appl. No.: 17/286,531

(22) PCT Filed: Oct. 17, 2019
(86) PCT No.: PCT/US2019/056805

§ 371 Date: Apr. 19, 2021

Related U.S. Application Data
(60) Provisional application No. 62/747,042, filed on Oct. 17, 2018.

(51) Int. Cl. A61F 9/04; B32B 27/12; B32B 5/02; A42B 1/04; A41D 31/12; A61K 9/00

(52) U.S. Cl. CPC A61F 9/045; B32B 27/12; B32B 5/022; B32B 2535/00; A41D 31/125; A61K 9/0014; B32B 2437/04; A42B 1/04

(57) ABSTRACT

An apparel system for applying topical treatments includes an article of apparel configured to cover and interface with a skin surface of a human body, the article of apparel including an oleophobic and moisture wicking layer. A method for applying a topical treatment is also disclosed.

### CROSS-REFERENCE TO RELATED APPLICATIONS

This application claims priority to U.S. provisional application No. 62/747,042 filed on Oct. 17, 2018 incorporated herein by reference in its entirety.

### BACKGROUND OF THE INVENTION

Patients that require topical treatments applied to the skin and other body surfaces often have a hard time keeping the topical ointment in place. For example, when treating psoriasis, this issue is particularly problematic when the psoriasis happens on scalp or skin areas near joins. For psoriasis that happens near joints (e.g., elbow, knee, shoulder, etc.), the applied topical ointment can be easily rubbed off by the clothing because of frequent movements of the joints. For psoriasis that happens on scalp, when the patient is asleep, the applied topical ointment can easily rub on sheets and pillows. When the patient places their head on the pillow and the medication unavoidably smears onto the pillow, the efficacy of the medication is decreased and this creates an unwanted mess on the beddings. Further, when patients with psoriasis, eczema or other skin conditions treated with topical medications apply their medication to their affected body parts, as soon as the patient places clothing or pajamas atop the medication, it rubs off or gets absorbed into the clothing or pajamas. Not only does the patient lose the effect of the medication, but the clothing and pajamas can become stained or ruined. Additionally, throughout the day when it could be desirable to apply a topical treatment, it would be beneficial for patients to have normal freedom of movement without disturbing the topical treatment or transferring it to other surfaces

Thus, what is needed in the art is a system and method that can assist with keeping topical treatments in place without limiting normal patient activity or unintentionally transferring the topical treatments to other surfaces.

### SUMMARY OF THE INVENTION

In one embodiment, an apparel system includes an article of apparel configured to cover and interface with a skin surface of a human body, the article of apparel comprising an oleophobic and moisture wicking layer. In one embodiment, the apparel system includes a first layer of the article of apparel configured to contact the skin surface during use. In one embodiment, the apparel system includes a second layer of the article of apparel comprising the water repelling and moisture wicking layer. In one embodiment, the second layer comprises a plurality of micropores having a diameter between 0.1 to 100 micrometers. In one embodiment, the second layer is hydrophobic and oleophobic. In one embodiment, the second layer comprises one of an expanded polytetrafluoroethylene membrane, a microporous polyurethane membrane, a polyester membrane, and an oleophobic PTFE membrane. In one embodiment, the second layer comprises a non-woven thermoplastic having a meltpoint lower than a meltpoint of the first layer. In one embodiment, the second layer comprises a bonding affinity to the first layer. In one embodiment, the apparel system includes a third layer of the article of apparel comprising an elastic material. In one embodiment, the third layer is configured adjacent to the second layer and opposite the first layer. In one embodiment, the second layer comprises a non-woven thermoplastic having a meltpoint lower than a meltpoint of the third layer. In one embodiment, the second layer comprises a bonding affinity to the third layer. In one embodiment, the article of apparel is one of a cap, shirt, sleeve, glove, pants, sock, undergarment and a wrap. A cap includes the apparel system and further includes a dome, the dome covering, at least partially, a top portion of a user's head, the dome having a lower circular end; and a cuff, the cuff has a top circular end and a lower circular end, the top circular end of the cuff is connected to the lower circular end of the dome; wherein the dome comprises the water repelling material and the moisture wicking material. In one embodiment, the cuff is configured to cover both ears of a user during use. In one embodiment, the cuff further is configured to cover both eyes of a user during use. In one embodiment, the apparel system includes a first strip having a first base connected to a rear center of the cuff, the first strip configured to extend from a rear center of the cuff and wrap around a left hemisphere of a user's head; and a second strip comprising a second base connected to the rear center of the cuff, the second strip configured to extend from the rear center of the cuff and warp around a right hemisphere of the user's head. In one embodiment, the first strip has a first front end disposed on an opposite end to the first base and the second strip has a second front end disposed on an opposite end to the second base. In one embodiment, the first front end includes an attachment mechanism, the second front end includes an attachment mechanism.

A method for applying a topical treatment includes the steps of applying the topical treatment to a portion of a user's skin, and occluding the topical treatment by covering and interfacing the portion of the user's skin with an article of apparel comprising an oleophobic material configured to contact the user's skin and configured adjacent to a moisture wicking material.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIGS. 1A and 1B are diagrams of a 3-layer system used for apparel according to one embodiment.

FIGS. 2A-2G are apparel items according to various embodiment. FIG. 2A is a shirt according to one embodiment, FIG. 2B is a sleeve according to one embodiment, FIG. 2C is a glove according to one embodiment, FIG. 2D is pants according to one embodiment, FIG. 2E is a sock according to one embodiment, FIG. 2E is a sock according to one embodiment, FIG. 2F is an undergarment according to one embodiment, and FIG. 2G is a wrap according to one embodiment.

FIG. 3 is a flow chart of a method for applying a topical treatment according to one embodiment.

FIG. 4 is a schematic diagram of a multilayer material for a cap according to one embodiment.

FIG. 5 is a multilayer cap according to one embodiment.

FIG. 6 is a multilayer cap according to one embodiment.

FIG. 7 is a multilayer cap according to one embodiment.

FIG. 8 is a multilayer cap according to one embodiment.

FIG. 9 shows an assembly of multilayer caps according to one embodiment.

FIG. 10 is a multilayer cap according to one embodiment.

FIG. 11 is a multilayer cap according to one embodiment.

FIG. 12 is a multilayer cap according to one embodiment.

FIG. 13A is a multilayer cap according to one embodiment and FIG. 13B is a multilayer cap according to one embodiment.

FIG. 14 is a multilayer cap according to one embodiment.

FIG. 15A is a multilayer cap according to one embodiment. FIG. 15B is a multilayer cap according to one embodiment.

FIG. 16A is a multilayer cap according to one embodiment, and FIG. 16B is a multilayer cap according to one embodiment.

FIG. 17 is a multilayer cap according to one embodiment.

FIG. 18 is a multilayer cap according to one embodiment of the disclosure.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in apparel systems and methods for enhancing the application of topical treatments. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is are apparel systems and methods for enhancing the application of topical treatments.

Embodiments of the apparel system and method described herein enhance the penetration of topical medication using the concept of occlusion therapy. In human skin, the permeability barrier lies in the stratum corneum. In inflammatory skin conditions, the permeability barrier is disrupted and thus leaves the skin susceptible to irritants. Occlusion provides better penetration and delivery of the topical medication. Occlusion can actually improve the penetration of medication in certain instances by 100 fold or more. Articles of apparel disclosed herein provide an occlusive microenvironment to aid in healing of diseased skin. The same principles hold true for patients with atopic dermatitis and other skin conditions. In certain instances, the article of apparel according to the embodiments described herein can be a treatment method itself without any medication. The apparel produces certain levels of therapeutic effect because of the microenvironment created (e.g., temperature, moisture level, etc.) which enhances the blood circulation and immune responses.

Certain embodiments may utilize a barrier textile including but not limited to multi-layer, bonded, coated or laminated textiles with a protective barrier material to prevent the migration of medications and ointments from the skin beyond the barrier textile. For example, in a three layer embodiment, the first layer (in contact with skin) can be a fabric layer that allows moisture vapor and air through. Ointment and creams are repelled by this layer, though some moisture may be held in this layer. This layer can be made from synthetic materials such as nylon/spandex, or natural fibers such as cotton/lyocell. This later serves as a soft layer next to the skin that allows moisture through to keep the patient comfortable. The second layer (middle layer) is a membrane layer that allows moisture vapor through, and repels ointments and creams. This layer can be made with for example PTFE, polyurethane, or similar barrier material. The purpose of this layer is to act as a barrier and repel medication and not permit medication to be absorbed into the textile. This layer also serves to protect the patient's clothing, bedding etc. and to provide occlusion therapy, which will allow increased penetration of the medication through the layers of the skin. The third layer (exterior layer) is a fabric layer that allows moisture vapor and air through, and adds a soft and comfortable feel to the apparel item. A two layer embodiment includes a first membrane layer (in contact with the skin) that lets moisture through and repels ointment and medication cream, and is made of PTFE or another similar material. This layer acts as a barrier and repels medication and not permit medication to be absorbed into the textile. The second layer (exterior layer) is a fabric layer that lets moisture vapor though, and can be made of synthetic materials such as nylon or spandex, or natural fibers such as cotton or lyocell. This layer also provides a soft and comfortable feel for the user.

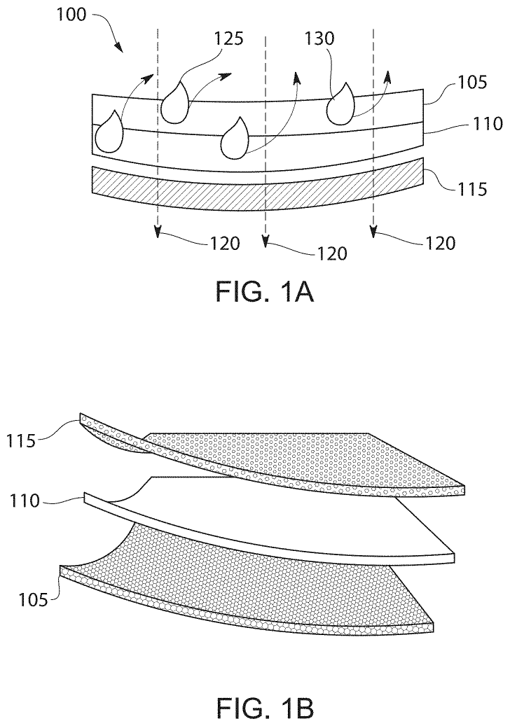

With reference now to FIGS. 1A and 1B, an apparel system is described according to one embodiment. The apparel system includes layers 100 that can be assembled as an item of apparel to cover and interface with a skin surface of a human body. A first layer 105 provides comfortable contact with the skin. The system includes a second layer 110 having a water repelling and moisture wicking material 110. In one embodiment, the second layer has micropores having a diameter between 0.1 to 100 micrometers. The second layer can be hydrophobic, oleophobic, or include one or more of an expanded polytetrafluoroethylene membrane, a microporous polyurethane membrane, a polyester membrane, and an oleophobic PTFE membrane. The second layer can include a non-woven thermoplastic having a meltpoint lower than a meltpoint of the first layer and a bonding affinity to the first layer. A third layer 115 of the article of apparel can include an elastic material. The third layer can be configured adjacent to the second layer and opposite the first layer. The second layer can have a non-woven thermoplastic having a meltpoint lower than a meltpoint of the third layer. The second layer can also include a bonding affinity to the third layer.

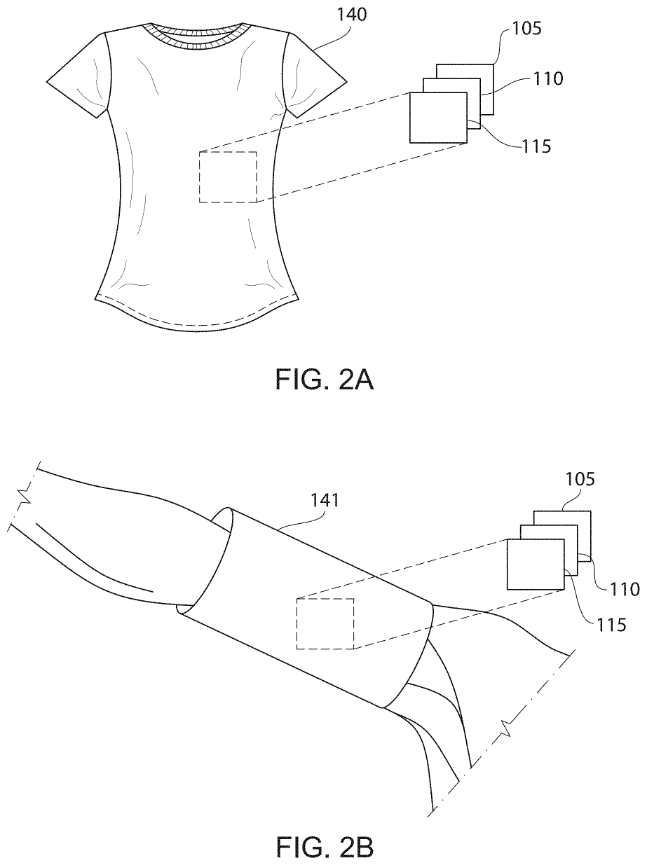

With reference now to FIGS. 2A-2G, the article of apparel can be one of a shirt 140, sleeve 141, glove 142, pants 143, sock 144, undergarment 145, wrap 146 or another item of apparel known in the art made of a flexible material and configured to cover and interface with a surface of a user's skin. With reference also to FIG. 1B and according to various apparel embodiments, the multilayer material 100 includes three layers, a first layer 105, a second layer 110, and a third layer 115. In one embodiment, the first layer 105 can be a skin or body contacting layer. The second layer 110 is a layer that is both hydrophobic (i.e., water repelling) and oleophobic (i.e., oil repelling). The second layer further provides moisture wicking properties. The third layer 115 can be an exterior layer, a layer disposed away from the scalp. The first layer 105 would be a scalp-contact layer that is soft against the skin. In one embodiment, the first layer may include nylon/spandex blend. A natural fiber similar to fine cotton such as lyocell can be used. The second layer 110 can be a water 125 repelling, oil 130 repelling, and moisture 120 wicking membrane layer. In one embodiment, the second layer 110 can be a microporous membrane or a non-microporous layer that has moisture 120 wicking ability. The second layer 110 allows the moisture 120 to penetrate through, creating a moisture 120 wicking property. The second layer 100 allows the covered scalp to be dry, because it does not create a sweating environment. In certain embodiments the second layer is not microporous but retains the same properties described herein. Those having skill in the art will recognize that various types, shapes and forms of apparel can be implemented according to the principals described herein for embodiments of the invention, and apparel items are now limited to the exemplary examples provided herein.

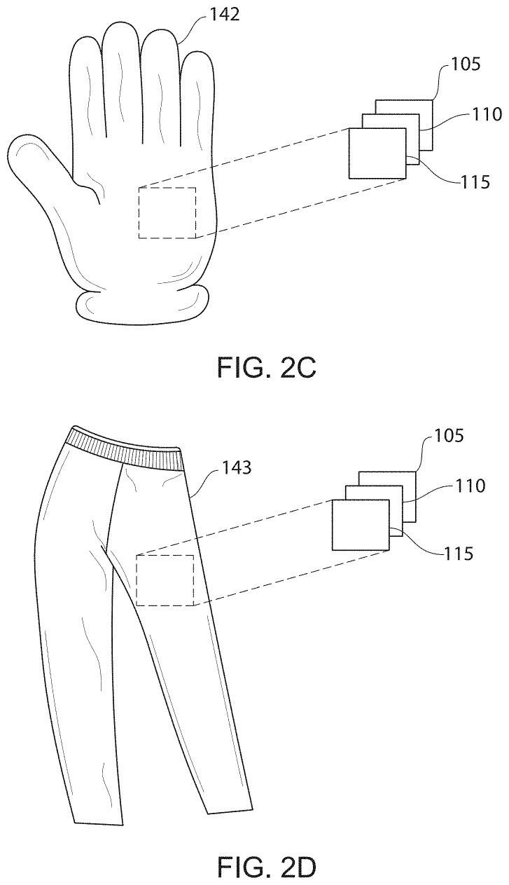

A method 10 for applying a topical treatment is described in FIG. 3 according to one embodiment. The method 10 includes the steps of applying the topical treatment to a portion of a user's skin 12, and occluding the topical treatment by covering and interfacing the portion of the user's skin with an article of apparel comprising a water repelling and moisture wicking layer.

Accordingly, the second layer 110 repels the water droplet 125 and repels the oil droplet 130. This means the second layer 110 is both hydrophobic and oleophobic. In one embodiment, the second layer 110 creates a comfortable environment for the skin by balancing the moisture 120 wicking property and water 125 repelling property. In one embodiment, this can be achieved by creating a layer with micro-pores. By selecting an appropriate diameter of the micro-pores, the balance between the moisture wicking and water repelling properties can be achieved. Micro-pores may have a diameter between 0.1 to 100 micrometers. In one embodiment the second layer 110 can be expanded polytetrafluoroethylene (PTFE) membrane, a microporous polyurethane membrane, polyester membrane, and oleophobic PTFE membrane. In one embodiment, the second layer 110 can be a material described in U.S. Pat. No. 9,901,128. The specification of the U.S. Pat. No. 9,901,128 is hereby incorporated in its entirety.

The third layer 115 can be an external layer, disposed away from the skin. In one embodiment, the third layer 115 may include nylon/spandex blend. In some embodiments, each of the first layer 105, the second layer 110, and the third layer 115 can be a piece of garment composed with fibers made from appropriate materials.

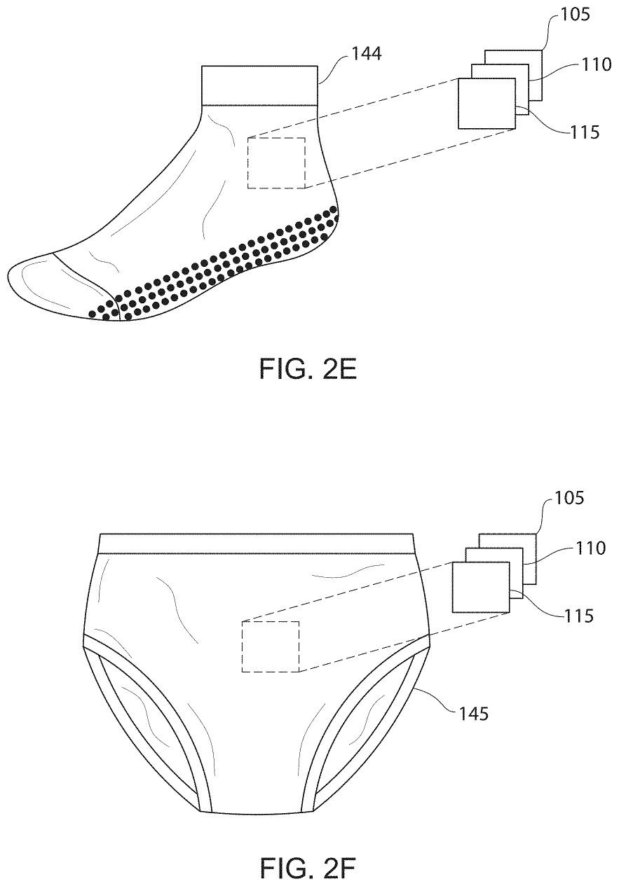

One particular apparel item will now be described in detail, although concepts described can apply to several different types of apparel. With reference now to FIG. 4 and according to one embodiment, a multilayer cap 200 is shown. The cap 200 includes a dome 205. The dome 205 covers a user's head. The dome 205 is breathable. In one embodiment the dome 205 is made with the multilayer material 100. In another embodiment, the dome 205 is made with MEDTEXTRA. In one embodiment, the dome 205 is formed with lightweight knit. The cap 200 includes a cuff 210. The cuff 210 circumscribes a user's head. The cuff 210 can be breathable. In one embodiment, the cuff 210 is made with elastic material such that it secures the cap 200 on the user's head. In one embodiment the cuff 210 is made with the multilayer material 100. In another embodiment, the cuff 210 is made with MEDTEXTRA. In one embodiment, the cuff 210 is formed with long loop knit. In one embodiment the cuff 210 is a 2.5 inch rib knit cuff. The cuff 210 may or may not cover the user's ears. In one embodiment, the cuff 210 can be above the ear design. In another embodiment, the cuff 210 can be below the ear design.

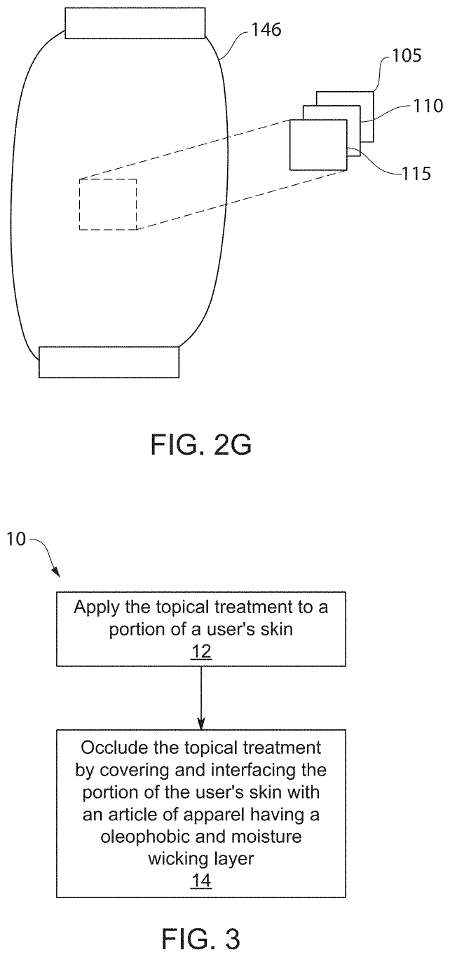

With reference now to FIG. 5 is a multilayer cap 300 according to one embodiment is shown. The cap 300 includes a dome 305. The dome 305 covers a top portion of a user's head. The dome 305 is breathable. In one embodiment the dome 305 is made with the multilayer material 100. In another embodiment, the dome 305 is made with MEDTEXTRA. In one embodiment, the dome 305 is formed with lightweight knit.

The cap 300 includes a cuff 310. The cuff 310 circumscribes a user's head. The cuff 310 can be breathable. In one embodiment, the cuff 310 is made with elastic material such that it secures the cap 300 around the user's head. In one embodiment the cuff 310 is made with the multilayer material 100. In another embodiment, the cuff 310 is made with MEDTEXTRA. In one embodiment, the cuff 310 is formed with long loop knit. As shown in FIG. 5, the cuff 310 includes an ear cover portion 315. The ear cover portion 315 may cover one or both of a user's ears. The ear cover portion 315 may be in a continuous knit, e.g., rib knit, with the cuff 310.

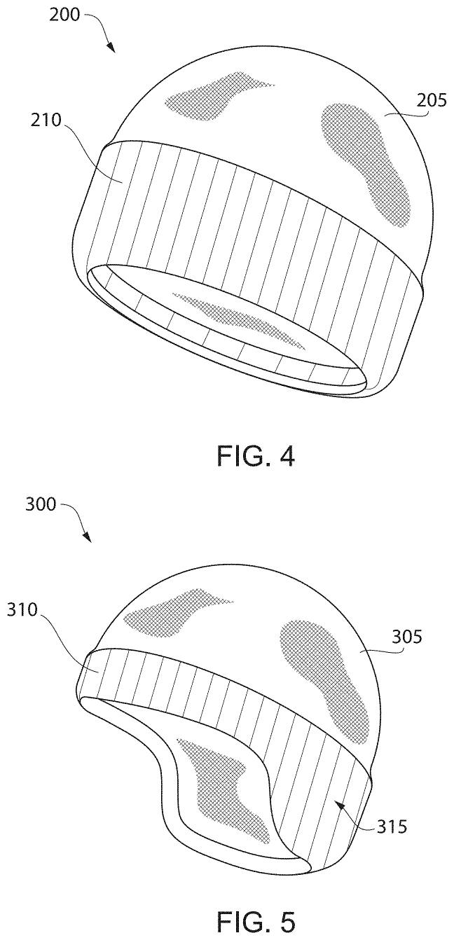

With reference now to FIG. 6 and according to one embodiment, a multilayer cap 400 is shown. The cap 400 includes a dome 405. The dome 405 covers a top portion of a user's head. The dome 405 is breathable. In one embodiment, the dome 405 is made with the multilayer material 100. In another embodiment, the dome 405 is made with MEDTEXTRA. In one embodiment, the dome 405 is formed with lightweight knit. The cap 400 includes a cuff 410. The cuff 410 circumscribes a user's head. The cuff 410 can be breathable. In one embodiment, the cuff 410 is made with elastic material such that it secures the cap 400 on the user's head. In one embodiment the cuff 410 is made with the multilayer material 100. In another embodiment, the cuff 410 is made with MEDTEXTRA. In one embodiment, the cuff 410 is formed with long loop knit. In one embodiment the cuff 410 is a rib knit cuff.

Two strips 415a and 415b are connected to the cuff 410. As shown in FIG. 6, a single strip 415 is in an elongated shape with a wider base connected to a rear center of the cuff 410. A first strip 415a extends from a rear center of the cuff 410 about a left hemisphere of a user's head. A second strip 415b extends from a rear center of the cuff 410 about a right hemisphere of the user's head. The strips 415a and 415b can be elastic. The strips 415a and 415b may have an attachment means at the front end to secure the strips 415a, 415b around the head. The strips 415a, 415b prevent the scalp topical medication from leaking or squeezing out. The cuff 410 may or may not cover the user's ears. In one embodiment, the cuff 410 can be above the ear design. In another embodiment, the cuff 410 can be below the ear design.

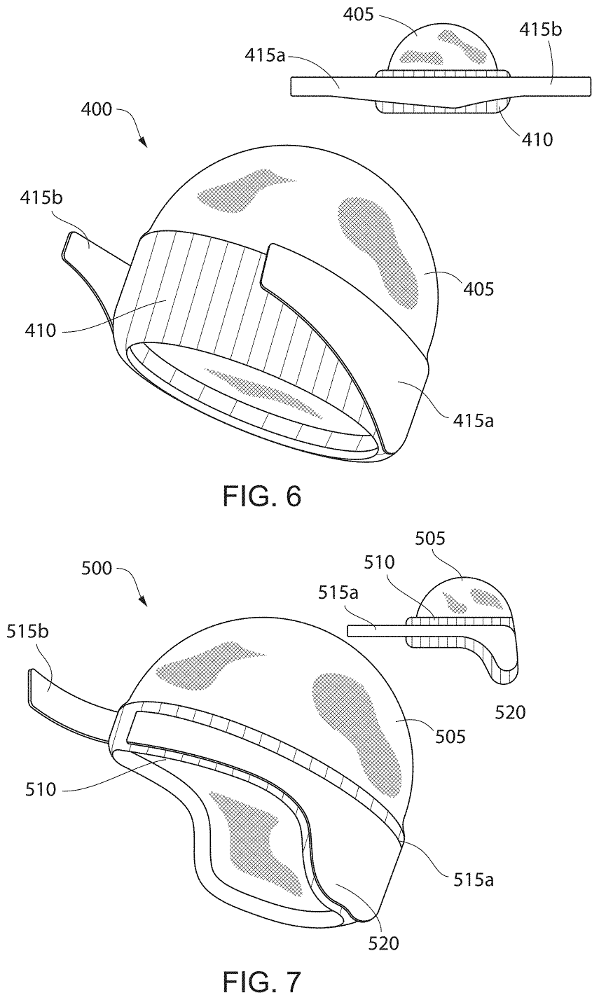

With reference now to FIG. 7, a multilayer cap 500 according to one embodiment. The cap 500 includes a dome 505. The dome 505 covers a top portion of a user's head. The dome 505 is breathable. In one embodiment, the dome 505 is made with the multilayer material 100. In another embodiment, the dome 505 is made with MEDTEXTRA. In one embodiment, the dome 505 is formed with lightweight knit. The cap 500 includes a cuff 510. The cuff 510 circumscribes a user's head. The cuff 510 can be breathable. In one embodiment, the cuff 510 is made with elastic material such that it secures the cap 500 around the user's head. In one embodiment the cuff 510 is made with the multilayer material 100. In another embodiment, the cuff 510 is made with MEDTEXTRA. In one embodiment, the cuff 510 is formed with long loop knit.

The cuff 510 includes an ear cover portion 520. The ear cover portion 520 may cover one or both of a user's ears. The ear cover portion 520 may be in a continuous knit, e.g., rib knit, with the cuff 510. Two strips 515a and 515b are connected to the cuff 510. A single strip 515 is in an elongated shape with a wider base connected to a rear center of the cuff 510. The base of the strip 515 covers the ear cover portion 520 of the cuff 510. A first strip 515a extends from a rear center of the cuff 510 about a left hemisphere of a user's head. A second strip 515b extends from a rear center of the cuff 510 about a right hemisphere of the user's head. The strips 515a and 515b can be elastic. The strips 515a and 515b may have an attachment means, e.g., Velcro, at the front end to secure the strips 515a, 515b around the head. The strips 515a, 515b prevent the scalp topical medication from leaking or squeezing out.

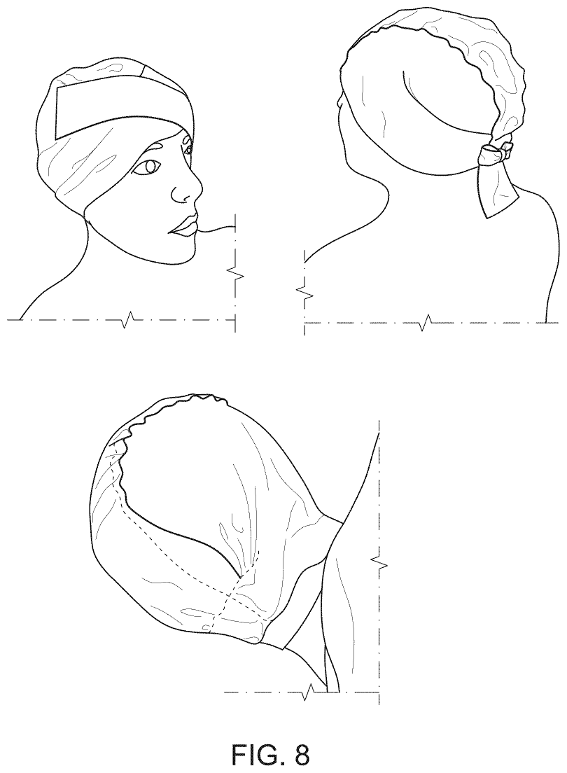

With reference now to FIG. 8, a multilayer cap according to one embodiment is shown. The cap includes a dome and two strips. Each of the strips extends from a rear center extending towards the front. The two strips crosses each other at the forehead. The front end of the strips include attachment means, e.g., Velcro, to secure the cap on a user's head. In one embodiment, the cap covers the nape. In one embodiment, the back of the cap has an elastic knot to secure the cap.

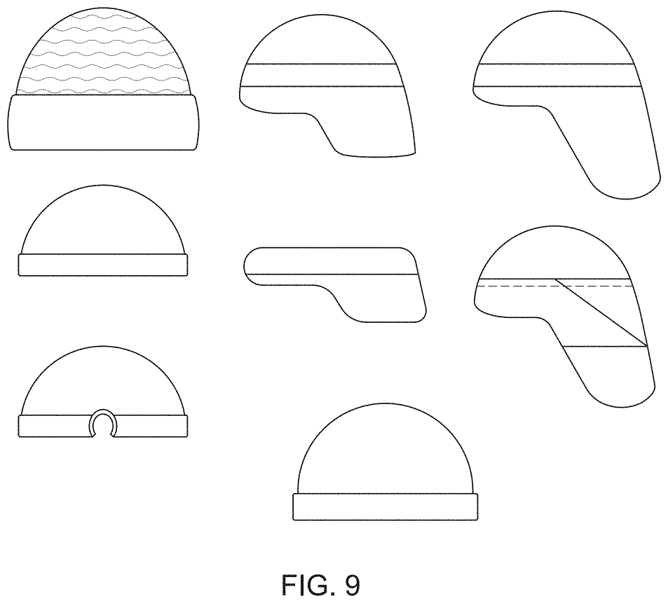

With reference now to FIG. 9, an assembly of multilayer caps according to one embodiment is shown. Various embodiments of caps are shown in FIG. 7. In some embodiments, the caps is above the ear (not covering the ear). In some embodiments, the caps cover the ears. In some embodiment, the cap includes one open notch at each side to accommodate the ear. In some embodiments, the cap covers the nape.

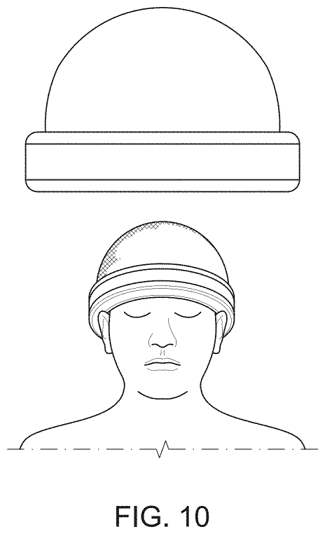

FIG. 10 shows a multilayer cap according to one embodiment of the disclosure. The cap in FIG. 8 includes a dome and a cuff. The cuff is designed not to cover the ears of the user.

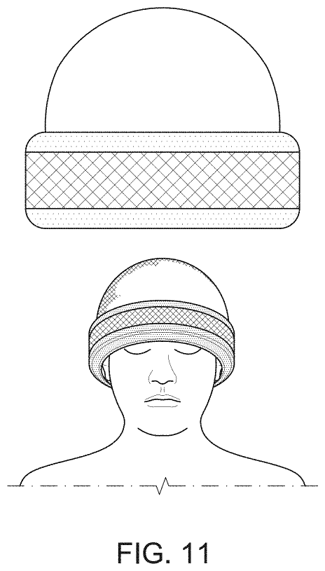

FIG. 11 is a multilayer cap according to one embodiment of the disclosure. The cap includes a dome and a cuff. The cuff in FIG. 9 is designed to cover the ears of the user.

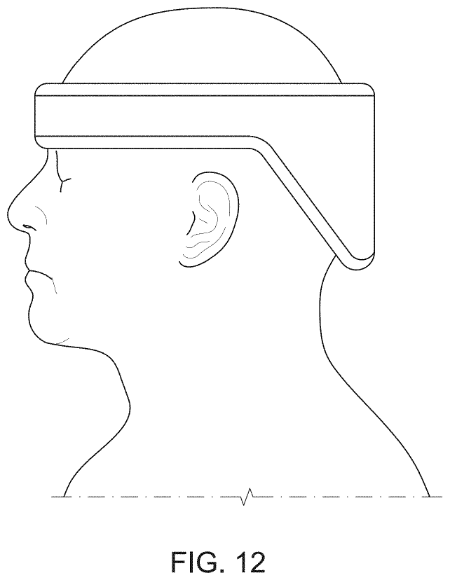

FIG. 12 is a multilayer cap according to one embodiment of the disclosure. The cap includes a dome and a cuff. The cuff has a wider coverage at the back of the head. The cuff is designed to cover a nape of a user, but not the ears.

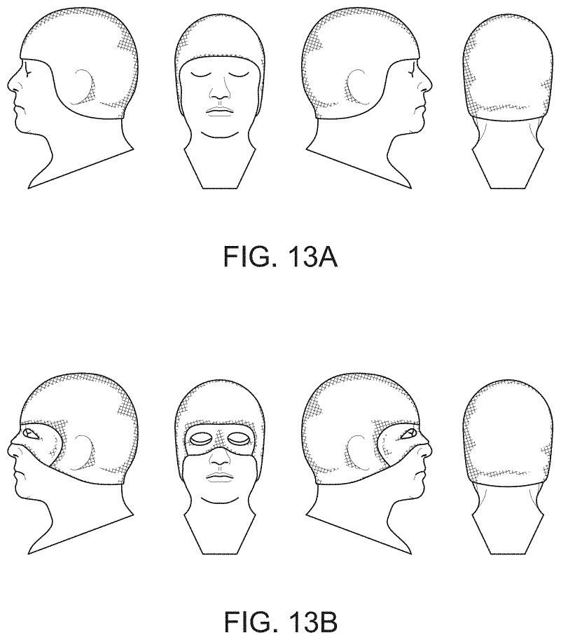

FIG. 13A is a multilayer cap according to one embodiment of the disclosure.

The cap covers the ears and the nape of the user.

FIG. 13B is a multilayer cap according to one embodiment of the disclosure.

The cap covers the ears, the nape, and the eyes of the user.

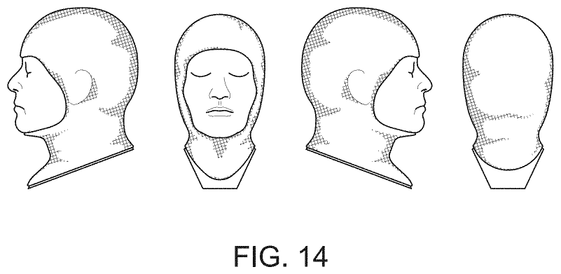

FIG. 14 is a multilayer cap according to one embodiment of the disclosure. The cap covers the ears, the nape, and the neck. The cap does not cover the eyes.

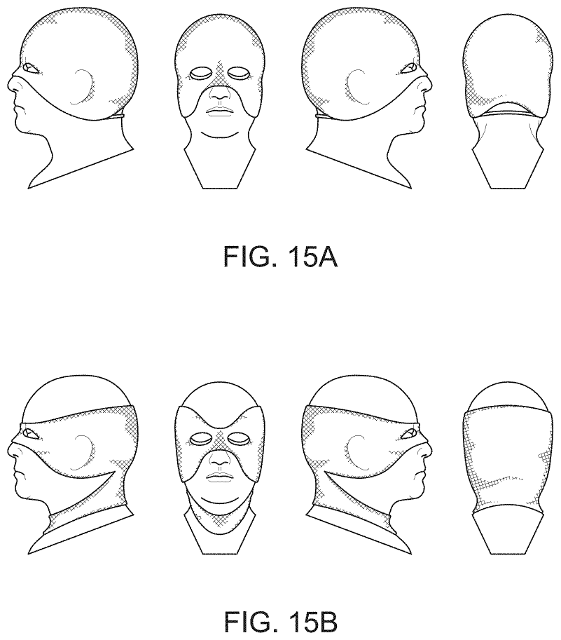

FIG. 15A is a multilayer cap according to one embodiment of the disclosure. The cap covers the eyes, the nape, and the ears. The cap further covers the nose, at least partially. The cap further covers the cheeks, at least partially.

FIG. 15B is a multilayer cap according to one embodiment of the disclosure. The cap covers the eyes, the ears, the nape, and partially the neck. The cap does not cover a top hemisphere of the user's head.

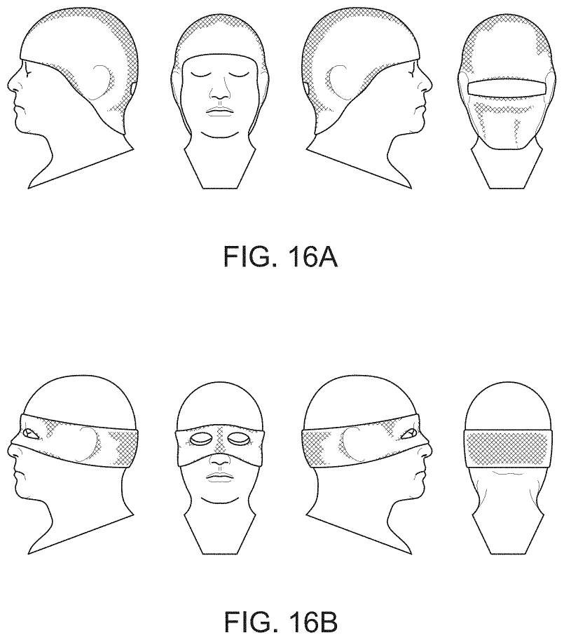

FIG. 16A is a multilayer cap according to one embodiment of the disclosure. The cap covers the ears and an upper back of the neck of the user. The cap has an opening at the nape area of the user.

FIG. 16B is a multilayer cap according to one embodiment of the disclosure. The cap is a circular bend structure, covering the eyes, ears, and nape of the user.

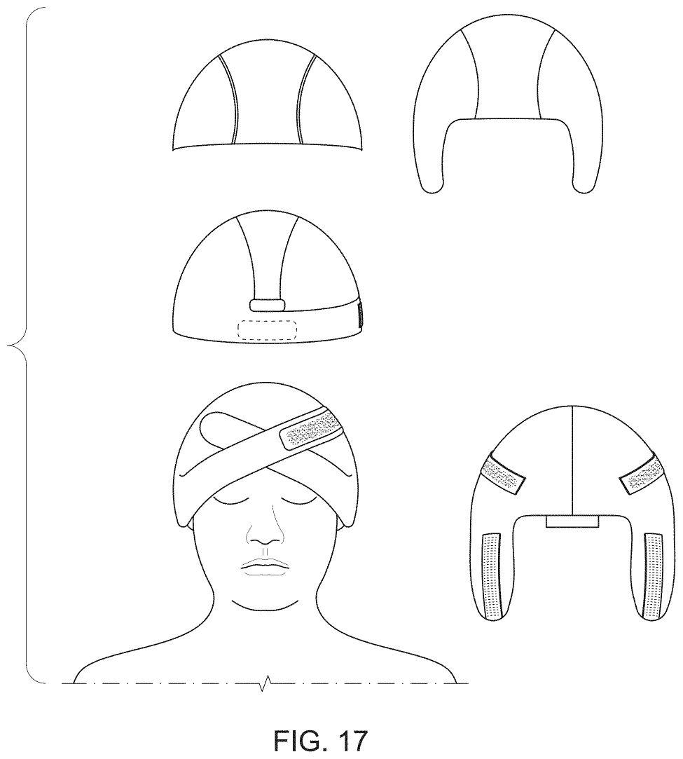

FIG. 17 is a multilayer cap according to one embodiment of the disclosure. The cap includes a dome, a cuff, and two strips. The two strips extends from a rear side of the cap toward a front side of the cap. The two strips crosses at a user's forehead. The two strips include Velcro as security means to secure the cap on the user's head.

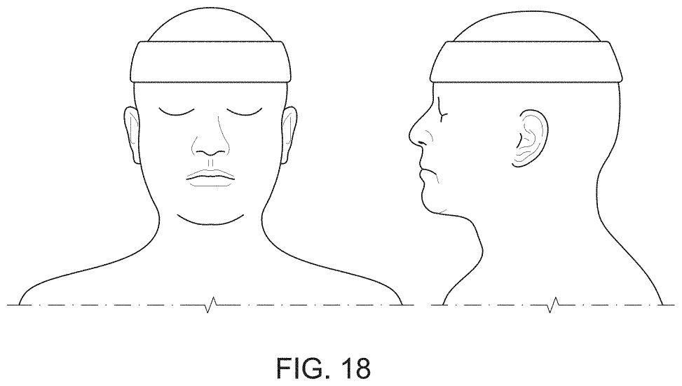

FIG. 18 is a multilayer cap according to one embodiment of the disclosure. The cap covers a nape but not the ears or the eyes of the user. The cap includes a cuff in a form of an elastic bend circulating a user's head.

In some embodiments, multilayer head caps are designed for patient with scalp psoriasis. Patients use topical medications to treat their psoriatic plaques. Topical medications include: clobetasol foam, clobetasol solution, mineral oil, general oil/petroleum based products, shampoos, salicylic acid, and some combinations of the above mentioned ingredients. In one embodiment, the multilayer head cap is designed with specific geometry to keep the topical ointment in place. The geometry of the multilayer head cap also allows patients to sleep comfortably without creating unwanted mess to the beddings.

In one embodiment, the multilayer head cap is made with a material that has three layers. The first layer would be a scalp-contact layer that would be a soft against the skin. In one embodiment, the first layer may include a nylon/spandex blend or a natural fiber such as a Tencel blend. The second layer can be a repellant/membrane layer. In one embodiment, the second layer can be a microporous membrane that has moisture wicking ability such that it does not create a sweating environment. In another embodiment, the second layer is oleophobic and/or hydrophobic. In one embodiment the second layer can be expanded polytetrafluoroethylene (PTFE) membrane, a microporous polyurethane membrane, polyester membrane, and oleophobic PTFE membrane. The third layer is an external layer. In one embodiment, the third layer can be a spandex, polyester, natural fibers such as Tencel, and/or nylon blend layer.

In one embodiment, to use the caps, patients would put on their scalp medication and then cover their heads with the caps. The cap is designed to keep the medication in place and prevent smearing. In some embodiments, the cap can cover the nape of the neck. The cap increases the occlusion of the medication which in turn increases the efficacy of the treatment.

It is specifically noted the embodiments disclosed herein are not limited to psoriasis. Any treatment involving topical medication can benefit from the embodiments disclosed herein.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An apparel system for applying a topical treatment comprising:
an article of apparel configured to cover and interface with a skin surface of a human body, the article of apparel comprising an oleophobic and moisture wicking layer.

2. The apparel system of claim 1 further comprising:
a first layer of the article of apparel configured to contact the skin surface during use.

3. The apparel system of claim 2 further comprising:
a second layer of the article of apparel comprising the water repelling and moisture wicking layer.

4. The apparel system of claim 3, wherein the second layer comprises a plurality of micropores having a diameter between 0.1 to 100 micrometers.

5. The apparel system of claim 4, wherein the second layer is hydrophobic and oleophobic.

6. The apparel system of claim 2, wherein the second layer comprises one of an expanded polytetrafluoroethylene membrane, a microporous polyurethane membrane, a polyester membrane, and an oleophobic PTFE membrane.

7. The apparel system of claim 2, wherein the second layer comprises a non-woven thermoplastic having a meltpoint lower than a meltpoint of the first layer.

8. The apparel system of claim 7, wherein the second layer comprises a bonding affinity to the first layer.

9. The apparel system of claim 4 further comprising:
a third layer of the article of apparel comprising an elastic material.

10. The apparel system of claim 9, wherein the third layer is configured adjacent to the second layer and opposite the first layer.

11. The apparel system of claim 10, wherein the second layer comprises a non-woven thermoplastic having a meltpoint lower than a meltpoint of the third layer.

12. The apparel system of claim 11, wherein the second layer comprises a bonding affinity to the third layer.

13. The apparel system of claim 1 wherein the article of apparel is one of a cap, shirt, sleeve, glove, pants, sock, undergarment and a wrap.

14. A cap comprising the apparel system of claim 1, the cap further comprising:
a dome, the dome covering, at least partially, a top portion of a user's head, the dome having a lower circular end; and
a cuff, the cuff has a top circular end and a lower circular end, the top circular end of the cuff is connected to the lower circular end of the dome;
wherein the dome comprises the water repelling material and the moisture wicking material.

15. The cap of claim 14, wherein the cuff is configured to cover both ears of a user during use.

16. The cap of claim 14, wherein the cuff further is configured to cover both eyes of a user during use.

17. The cap of claim 14 further comprising:
a first strip having a first base connected to a rear center of the cuff, the first strip configured to extend from a rear center of the cuff and wrap around a left hemisphere of a user's head; and
a second strip comprising a second base connected to the rear center of the cuff, the second strip configured to extend from the rear center of the cuff and warp around a right hemisphere of the user's head.

18. The cap of claim 17, wherein the first strip has a first front end disposed on an opposite end to the first base and the second strip has a second front end disposed on an opposite end to the second base.

19. The cap of claim 18, wherein the first front end includes an attachment mechanism, the second front end includes an attachment mechanism.

20. A method for applying a topical treatment comprising:
applying the topical treatment to a portion of a user's skin; and
occluding the topical treatment by covering and interfacing the portion of the user's skin with an article of apparel comprising an oleophobic material configured to contact the user's skin and configured adjacent to a moisture wicking material.
